(19) United States
(12) Patent Application Publication
(10) Pub. No.: US 2020/0215025 A1
(43) Pub. Date: Jul. 9, 2020

(54) TREATMENT OF EXCITOTOXICITY

(71) Applicant: Norbio No. 1 Pty Ltd, Gordon, New South Wales (AU)

(72) Inventors: Graham KELLY, Gordon, New South Wales (AU); Benny EVISON, Gordon, New South Wales (AU); Gary HOUSLEY, Gordon, New South Wales (AU)

(21) Appl. No.: 16/647,091

(22) PCT Filed: Sep. 17, 2018
(86) PCT No.: PCT/AU2018/051014

§ 371 Date: Mar. 13, 2020

Related U.S. Application Data
(60) Provisional application No. 62/559,348, filed on Sep. 15, 2017.

(51) Int. Cl. A61K 31/353; A61K 9/02; A61K 9/00

(52) U.S. Cl. CPC A61K 31/353; A61K 9/0034; A61K 9/02

(57) ABSTRACT

The invention relates to methods for treating or minimising nervous system excitotoxicity, or a condition associated with excitotoxicity, through inhibition of calcium release and/or calcium uptake from a cell, in an individual utilising a compound according to general formula (1).

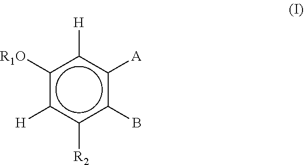

### FIELD OF THE INVENTION

The invention relates to treatment of diseases or conditions associated with excitotoxicity including acute conditions such as stroke and brain ischemia/reperfusion injury, central nervous system (CNS) trauma such as traumatic brain injury (TBI), and chronic neurodegenerative disorders.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a person skilled in the art.

Recent studies have established a connection between excitotoxicity and neuroinflammation whereby an abnormal neuroinflammatory response may mediate excitotoxic damage to neural tissue [Mc Dowell M L et al. 2011 Neurochem Int 59(2): 175-184]. Glial cells appear to be a key player as they are activated in response to excitotoxic lesions [Drouin-Ouellet J. et al. 2011 Glia 59(2):188-199]. They also release pro-inflammatory cytokines (TNF-α, IL-1 α, IL-1 β, IL-1RA, IL-2, IL-3 and IL-10) in response to immune cell signaling. These pro-inflammatory cytokines are understood to be relevant to neuronal injury in spinal cord injury and many chronic neurodegenerative diseases including, Alzheimer's disease, Parkinson's disease, and multiple sclerosis [Mc Dowell supra]. Further, excitotoxicity, especially as arises from excessive levels of glutamate, is a hallmark of reperfusion/ischemia injury, CNS trauma, neurotoxin, bacterial or viral invasion of neural tissue.

Glial cells are confined to the CNS and it is understood that in this anatomical compartment they interact with a variety of cells of non-neural origin, principally in response to pro-inflammatory signals received from immune cells such as mast cells, and signals received from other cells including glutamate signaling [Drouin-Ouellet supra]. This cellular network which is unique to the CNS suggests pro- and anti-inflammatory mechanisms of action and excitotoxic outcomes that are specific to the neural tissue and in particular the CNS [Cherry J D et al. 2014 J. Neuroinflammation 11:98; Drouin-Ouellet supra].

Glutamate excitotoxicity in the central nervous system reflects the dysregulation of glutamate release and re-uptake. Under pathophysiological conditions such as stroke/ischemic brain injury, the cell stress leads to excess release of glutamate from excitatory synapses, driving sustained activation of ionotropic (AMPA, Kainate and NMDA-types) glutamate receptors and metabotropic glutamate receptors (G protein-coupled receptors). This can lead to direct and secondary Ca²⁺ loading of neurons that triggers down-stream neuron pathology. The tissue stress also leads to loss of glutamate from neurons and astrocytes due to the run-down of the glutamate transporters, enhancing the extracellular glutamate signal [Mark L P et al. 2001, Am J Neuroradiol 22(10):1813-1824; Malarkey & Purpura 2008, Neurochem Int 52(1-2): 142-154].

McDowell supra discusses an in vitro neuroprotective effect of Genistein on motor neurons exposed to activated microglial cytokines. In one in vivo animal study, Genistein was found to be associated with neuroprotection in brain injury, where its activity was compared with that of related isoflavones with comparable anti-oxidant activity, where Genistein was found to provide greater neuroprotection, and hence the neuroprotection was apparently not related to an isoflavonoid-related antioxidant action but rather to some other unidentified biochemical activity specific to Genistein. [Trieu V N and Uckun F M 1999 Biochem Biophys Res Commun 19(3):685-688; Trieu V N et al. 1999 Radiat Res. 152(5):508-516.]

Genistein is a member of a large family of compounds generally known as isoflavonoids. While some of these compounds have been shown to have anti-inflammatory effects in non-neural tissue, many have been found not to be anti-inflammatory or to have much lesser anti-inflammatory activity. Confounding the problem is that those that are anti-inflammatory share chemical structures with those that have lesser anti-inflammatory activity [Hamalainen M et al. 2007 Mediators inflamm. Article ID 45673, 10 panes doi:10.1155/2007/45673; Zheng C et al. 2016 Sci. Rep. 6:31743; doi: 10.1038/srep31743].

Genistein has been studied in clinical trials for antineoplastic activity. The potentially valuable therapeutic opportunities of the compound have failed to translate into the clinic. One reason for this is that there is a question as to the susceptibility of Genistein and other isoflavonoids to Phase 1 and Phase 2 metabolic process with resulting decrease in potency and bioavailability. In fact, many isoflavonoid molecules are highly insoluble in water and are converted into water-soluble forms that are excreted from the body shortly after administration. Accordingly, the half-life of Genistein is short (about 4 to 6 hours).

### SUMMARY OF THE INVENTION

In one embodiment there is provided a method for treating or minimising excitotoxicity, or a condition associated with excitotoxicity in an individual including:
(i) providing an individual for whom treatment or minimisation of excitotoxicity is required;
(ii) administering a therapeutically effective amount of a compound of general Formula (I) to the individual

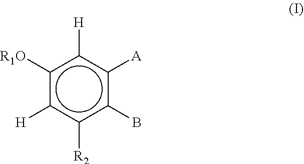

wherein

R₁ is H, or R_{A}CO where R_{A} is C₁₋₁₀ alkyl or an amino acid;

R₂ is H, OH, or R_{B} where R_{B} is an amino acid or COR_{A} where R_{A} is C₁₋₁₀ alkyl or an amino acid;

A and B together with the atoms between them form the group:

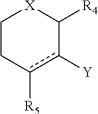

wherein

R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where R_{C} is C₁₋₁₀ alkyl, or CONHR_{E} where R_{E} is H, C₁₋₁₀ alkyl or an amino acid;

R₅ is substituted or unsubstituted aryl or substituted or unsubstituted heteroaryl;

X is O, N or S;

Y is

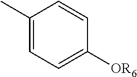

where R₆ is H, or C₁₋₁₀ alkyl; and

“ [symbol] ” represents either a single bond or a double bond;

wherein said administration minimises or treats excitotoxicity in the individual.

In another embodiment there is provided a method for treating or minimising excitotoxicity, or a condition associated with excitotoxicity in an individual including:
(iii) providing an individual for whom treatment or minimisation of excitotoxicity is required;
(iv) administering a therapeutically effective amount of a compound of general Formula (I) to the individual

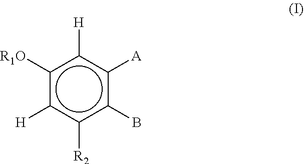

wherein

R₁ is H, or R_{A}CO where R_{A} is C₁₋₂₀ alkyl or an amino acid;

R₂ is H, OH, or R_{B} where R_{B} is an amino acid or OCOR_{A} where R_{A} is C₁₋₂₀ alkyl or an amino acid;

A and B together with the atoms between them form the group:

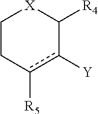

wherein

R₄ is H, COR_{D} where R_{D} is H, OH, C₁₋₂₀ alkyl or an amino acid, CO₂R_{C} where R_{C} is C₁₋₂₀ alkyl, COOH, or CONHR_{E} where R_{E} is H, C₁₋₂₀ alkyl or an amino acid;

R₅ is substituted or unsubstituted aryl or substituted or unsubstituted heteroaryl;

X is O, N or S;

Y is

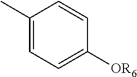

where R₆ is H, or OCR_{A} where R_{A} is C₁₋₂₀ alkyl or an amino acid; and

“ [symbol] ” represents either a single bond or a double bond;

wherein said administration minimises or treats excitotoxicity in the individual.

In certain embodiments, the compound of Formula (I) is:

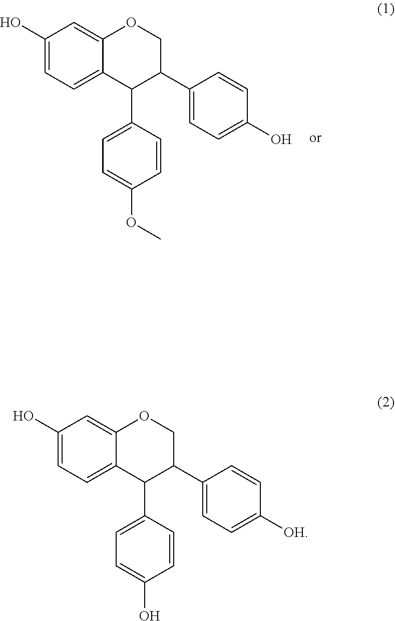 [SMILES: COc1ccc(C2c3ccc(O)cc3OCC2c2ccc(O)cc2)cc1 ; "or" ; Oc1ccc(C2COc3cc(O)ccc3C2c2ccc(O)cc2)cc1]

In some embodiments, the compounds may be present as racemic mixtures. In further embodiments, the compounds may be present as pairs of enantiomers, preferably in a 1:1 ratio. In other aspects, specific enantiomers will be favoured. In one embodiment, therefore, the present invention utilises any one of the following enantiomers:

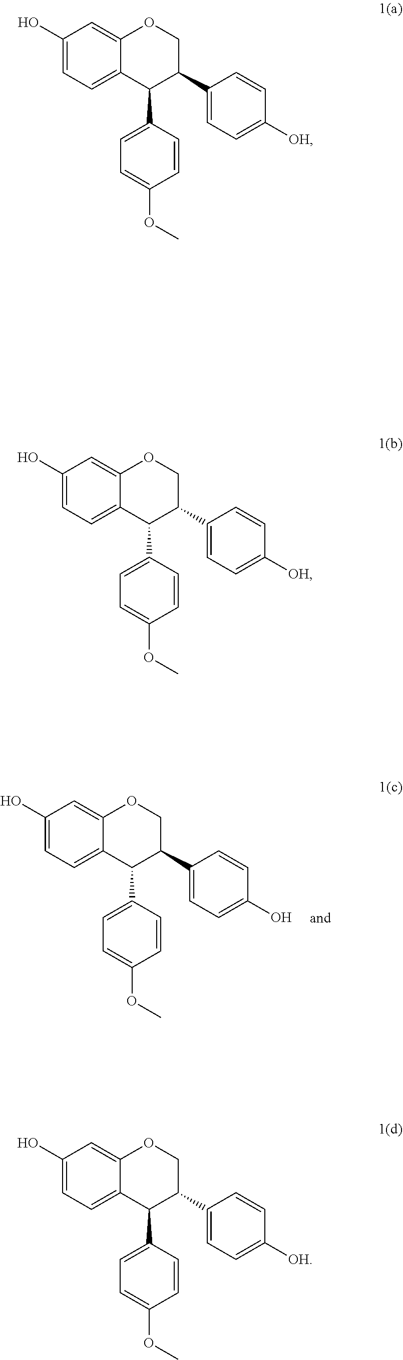 [SMILES: COc1ccc([C@H]2c3ccc(O)cc3OC[C@H]2c2ccc(O)cc2)cc1 ; COc1ccc([C@@H]2c3ccc(O)cc3OC[C@@H]2c2ccc(O)cc2)cc1 ; COc1ccc([C@@H]2c3ccc(O)cc3OC[C@H]2c2ccc(O)cc2)cc1 ; "and" ; COc1ccc([C@H]2c3ccc(O)cc3OC[C@@H]2c2ccc(O)cc2)cc1]

In one embodiment, the compound of the present invention is a pair of enantiomers of compound 1(a) and compound 1 (b), compound 1(a) and compound 1(c), compound 1(a) and compound 1(d), compound 1(b) and compound 1(c), compound 1(b) and compound 1(d), or compound 1(c) and compound 1(d). Preferably wherein the compound of the present invention is a pair of enantiomers, the pair of enantiomers comprises compound 1(a) and compound 1(b). One enantiomer in the pair of enantiomers may be enriched compared to the other enantiomer. Preferably, the pair of enantiomers are present in a 1:1 ratio.

In a preferred embodiment of the invention, the compound of the present invention is a specific enantiomer. In one embodiment, the compound of the present invention is

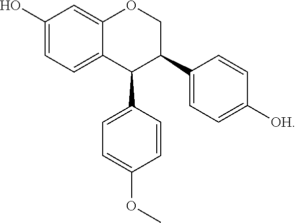 [SMILES: COc1ccc([C@H]2c3ccc(O)cc3OC[C@H]2c2ccc(O)cc2)cc1]

In another embodiment, the compound of the present invention is

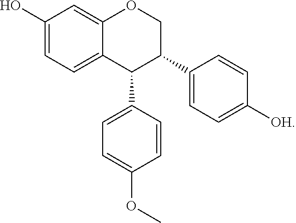 [SMILES: COc1ccc([C@@H]2c3ccc(O)cc3OC[C@@H]2c2ccc(O)cc2)cc1]

In another embodiment, the compound of the present invention is

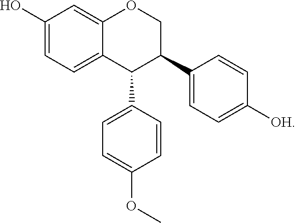 [SMILES: COc1ccc([C@@H]2c3ccc(O)cc3OC[C@H]2c2ccc(O)cc2)cc1]

In yet another embodiment, the compound of the present invention is

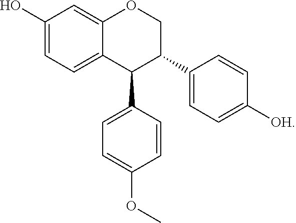 [SMILES: COc1ccc([C@H]2c3ccc(O)cc3OC[C@@H]2c2ccc(O)cc2)cc1]

In a preferred embodiment, the compound of the present invention is Compound 1(b). In a particularly preferred embodiment, the compound of the present invention is Compound 1(a).

In one embodiment, there is provided a method for inhibiting Ca²⁺ release from a cell including the step of providing a compound as described above to a cell, thereby inhibiting Ca²⁺ release from the cell. In another embodiment, there is provided a method for inhibiting Ca²⁺ uptake by a cell including the step of providing a compound as described above to a cell, thereby inhibiting Ca²⁺ uptake by the cell. Preferably the cell is a neuron. Preferably the compound is Compound 1 as described above. Preferably the inhibition of Ca²⁺ release by the cell, or inhibition of Ca²⁺ uptake by the cell prevents cell death or prevents excitotoxicity.

Thus in another embodiment there is provided a method for preventing cellular Ca²⁺ release and/or cellular Ca²⁺ uptake associated with excitotoxicity including the step of providing a compound as described above to a cell, thereby preventing cellular Ca²⁺ release and/or cellular Ca²⁺ uptake. Preferably the compound is Compound 1 as described above. Preferably the excitotoxicity arises from or is associated with reperfusion or ischemia. Preferably the excitotoxicity is associated with infarction of CNS tissue. Preferably the infarction arises from thrombosis.

In another embodiment there is provided a method for inhibiting the formation of an infarct in CNS tissue in an individual including the step of providing a compound as described above to an individual at risk of formation of an infarct in CNS tissue, thereby inhibiting the formation of an infarct in the individual. The individual may be at risk of, or have ischemia of CNS tissue. The individual may be at risk of thrombus formation. The individual may have had a stroke, or may be at risk for stroke. The method may result in a minimisation of the surface area of an infarct. The method may result in a minimisation of the volume of an infarct. Preferably the compound is Compound 1.

In another embodiment there is provided a compound of Formula (I), preferably Compound (1), for use in a method of minimising or treating excitotoxicity in an individual, or for minimising or treating a condition associated with excitotoxicity in an individual. The condition may be acute or chronic. An individual having an acute condition may have, or be at risk of, ischemia/reperfusion injury, a spinal cord injury, infarction, thrombosis, or stroke. An individual having a chronic injury may have, or be at risk of, a chronic neurodegenerative disease including, Alzheimer's disease, Parkinson's disease, or multiple sclerosis.

In the above described embodiments, a compound of Formula (I), preferably Compound 1, may be administered rectally.

In one embodiment there is provided a composition including:
an oleaginous base for use in a device for rectal, vaginal or urethral application;
a compound of Formula (I), preferably Compound 1.

### BRIEF DESCRIPTION OF THE FIGURES

FIG. 1 Representative traces of the cytosolic Ca²⁺ levels in HEK293 cells expressing the genetically encoded Ca²⁺ reporter (GCaMP5G). (A) Nx-1 (4 μM) block of carbachol (100 μM)—induced Ca²⁺ store release (in Ca²⁺ free solution at 200 s. (B) Block of Gq—PLC—IP₃ mediated Ca²⁺ store release by Nx-2 (4 μM), and of Ca²⁺ re-entry, as for Nx-1.

FIG. 2 Block of Ca²⁺ store release and Ca²⁺ re-entry by Nx-1 as described in FIG. 1 for HEK293 cells expressing the GCaMP5G Ca²⁺ reporter.

FIG. 3 Block of Ca²⁺ store release and Ca²⁺ re-entry by Nx-2 as described in FIGS. 1 and 2 for HEK293 cells expressing the GCaMP5G Ca²⁺ reporter.

FIG. 4 Dose response curves comparing the inhibition of Ca²⁺ store release (A) and Ca²⁺ re-entry (B) by Nx-1 and Nx-2. For Ca²⁺ store release, data for 0.1 μM of Nx-1 is normalised to 1.0. Ca²⁺ store release is virtually blocked at 4 μM for both Nx-1 and Nx-2, and the data points therefore overlay. The EC₅₀ is ˜2.0 μM for Nx-1 and ˜700 nM for Nx-2 for both Ca²⁺ store release and re-entry. Data are areas under the curves analysed using relative fluorescent units, normalised against the average of the DMSO control data (n=8-12 per concentration, mean±std.dev.).

FIG. 5 Box plot comparing the cerebral cortex surface infarct area of the core reference lesion at 2 hours post injury (2HPI, n=3) to the lesion at 5 days post injury (5 DPI) in mice treated with carrier only (n=6) or Nx-2 drug (100 mg/kg; n=6).

FIG. 6 Box plot comparing the infarct volumes of the core reference lesion at 2 hours post injury (2HPI, n=3) to the lesion at 5 days post injury (5 DPI) in mice treated with carrier only (n=6) or Nx-2 drug (100 mg/kg, n=6).

FIG. 7 Box plot comparing the cerebral cortex surface infarct area of the core reference lesion at 2 hours post injury (2HPI, n=3) to the lesion at 5 days post injury (5 DPI) in mice treated with carrier only (n=6) or Nx-1 drug (100 mg/kg; n=4).

FIG. 8 Box plot comparing the infarct volumes of the core reference lesion at 2 hours post injury (2HPI, n=3) to the lesion at 5 days post injury (5 DPI) in mice treated with carrier only (n=6) or Nx-1 drug (100 mg/kg, n=4).

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text. All of these different combinations constitute various alternative aspects of the invention.

As used herein, except where the context requires otherwise, the term “comprise” and variations of the term, such as “comprising”, “comprises” and “comprised”, are not intended to exclude further additives, components, integers or steps.

A. Compounds and Synthetic Methods

As discussed above, in one aspect the invention provides for uses of isoflavonoid compounds for minimising or treating excitotoxicity. The compounds and relevant synthetic methods are described below.

In one embodiment, the compound for use in the method of minimising or treating excitotoxicity is a compound of general Formula (I):

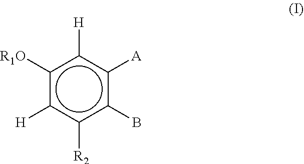

wherein

R₁ is H, or R_{A}CO where R_{A} is C₁₋₁₀ alkyl or an amino acid;

R₂ is H, OH, or R_{B} where R_{B} is an amino acid or COR_{A} where R_{A} is C₁₋₁₀ alkyl or an amino acid;

A and B together with the atoms between them form the group:

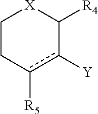

wherein

R₄ is H, COR_{D} where R_{D} is H, OH, C₁₋₁₀ alkyl or an amino acid, CO₂R_{C} where R_{C} is C₁₋₁₀ alkyl, COR_{E} where R_{E} is H, C₁₋₁₀ alkyl or an amino acid, COOH, COR_{C} where

R_{C} is C₁₋₁₀ alkyl, or CONHR_{E} where R_{E} is H, C₁₋₁₀ alkyl or an amino acid;

R₅ is substituted or unsubstituted aryl or substituted or unsubstituted heteroaryl;

X is O, N or S;

Y is

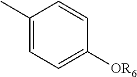

where R₆ is H, or C₁₋₁₀ alkyl; and

“ [symbol] ” represents either a single bond or a double bond.

In another embodiment, the compound for use in the method of minimising or treating excitotoxicity is a compound of general Formula (I):

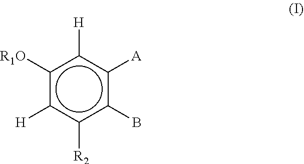

wherein

R₁ is H, or R_{A}CO where R_{A} is C₁₋₂₀ alkyl or an amino acid;

R₂ is H, OH, or R_{B} where R_{B} is an amino acid or OCOR_{A} where R_{A} is C₁₋₂₀ alkyl or an amino acid;

A and B together with the atoms between them form the group:

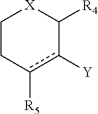

wherein

R₄ is H, COR_{D} where R_{D} is H, OH, C₁₋₂₀ alkyl or an amino acid, CO₂R_{C} where R_{C} is C₁₋₂₀ alkyl, COOH, or CONHR_{E} where R_{E} is H, C₁₋₂₀ alkyl or an amino acid;

R₅ is substituted or unsubstituted aryl or substituted or unsubstituted heteroaryl;

X is O, N or S;

Y is

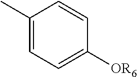

where R₆ is H, or R_{A}CO where R_{A} is C₁₋₂₀ alkyl or amino acid; and

“ [symbol] ” represents either a single bond or a double bond.

Preferably, X is O.

In another preferred embodiment, R₁ is H. In yet another preferred embodiment, R₂ is H. Preferably, R₄ is H.

In one preferred embodiment, R₅ is substituted aryl. Preferably, R₅ is aryl substituted with an alkoxy group. Preferably, the alkoxy group is methoxy. Preferably, the alkoxy substituent is in the 4-position. In another preferred embodiment, R₅ is aryl substituted with hydroxy. Preferably, the hydroxy substituent is in the 4-position.

In yet another embodiment, R₆ is H.

In preferred embodiments, the compound of Formula (I) is:

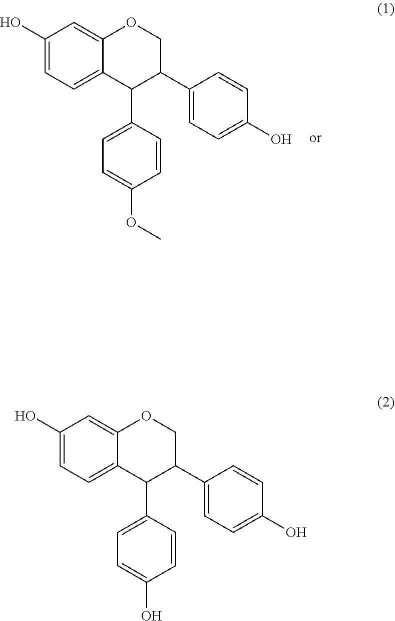 [SMILES: COc1ccc(C2c3ccc(O)cc3OCC2c2ccc(O)cc2)cc1 ; "or" ; Oc1ccc(C2COc3cc(O)ccc3C2c2ccc(O)cc2)cc1]

In a particularly preferred embodiment, the compound of Formula (I) is:

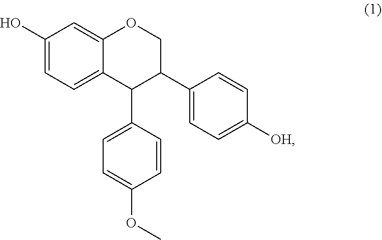 [SMILES: COc1ccc(C2c3ccc(O)cc3OCC2c2ccc(O)cc2)cc1]

otherwise known as triphendiol, or 3-(4-hydroxyphenyl)-4-(4-methoxyphenyl)chroman-7-ol or 3-(4-hydroxyphenyl)-4-(4-methoxyphenyl)-3,4-dihydro-2H-chromen-7-ol.

As used herein the term “alkyl” refers to a straight or branched chain hydrocarbon radical having from one to ten carbon atoms, or any range between, i.e. it contains 1, 2, 3, 4, 5, 6, 7, 8, 9 or 10 carbon atoms. The alkyl group is optionally substituted with substituents, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “C₁₋₂₀ alkyl” refers to an alkyl group, as defined above, containing at least 1, and at most 20 carbon atoms respectively, or any range in between (e.g. alkyl groups containing 2-5 carbon atoms are also within the range of C₁₋₂₀).

Preferably the alkyl groups contain from 1 to 5 carbons and more preferably are methyl, ethyl or propyl.

As used herein, the term “aryl” refers to an optionally substituted benzene ring. The aryl group is optionally substituted with substituents, multiple degrees of substitution being allowed.

As used herein, the term “heteroaryl” refers to a monocyclic five, six or seven membered aromatic ring containing one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl and substituted versions thereof.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterised and tested for biological activity.

The terms “optionally substituted” or “may be substituted” and the like, as used throughout the specification, denotes that the group may or may not be further substituted, with one or more non-hydrogen substituent groups. Suitable chemically viable substituents for a particular functional group will be apparent to those skilled in the art.

Examples of substituents include but are not limited to:

C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aminosulfonyl, acyloxy, alkoxycarbonyl, nitro, cyano or halogen.

The term “isoflavonoid” as used herein is to be taken broadly and includes isoflavones, isoflavenes, isoflavans, isoflavanones, isoflavanols and similar or related compounds. Some non-limiting examples of isoflavonoid core structures are shown below:

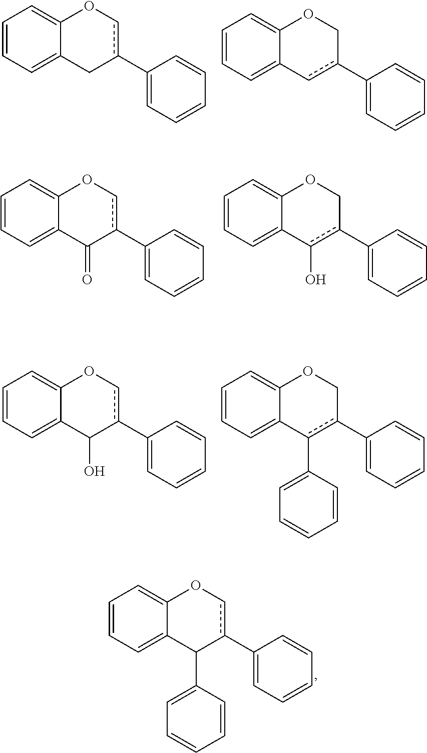

wherein “ [symbol] ” represents either a single bond or a double bond.

Methods for synthesis of the above described compounds are described in WO2005/049008 and references cited therein towards the synthesis, the content of which is incorporated herein by reference in entirety.

The compounds include all salts, such as acid addition salts, anionic salts and zwitterionic salts, and in particular include pharmaceutically acceptable salts as would be known to those skilled in the art. The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, tosylate, citrate, bicarbonate and carbonate. Pharmaceutically acceptable salts include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, para-toluenesulphonic, tartaric, trifluoroacetic, triphenylacetic, tricarballylic, salicylic, sulphuric, sulphamic, sulphanilic and succinic acid.

The term “pharmaceutically acceptable derivative” or “prodrug” refers to a derivative of the active compound that upon administration to the recipient is capable of providing directly or indirectly, the parent compound or metabolite, or that exhibits activity itself and includes for example phosphate derivatives and sulphonate derivatives. Thus, derivatives include solvates, pharmaceutically active esters, prodrugs or the like.

The preferred compounds of the present invention also include all derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

B. Formulation

The compounds of Formula (I) may be provided in the form of a pharmaceutical composition including at least one pharmaceutically acceptable excipient, especially for use in treatment or in the manufacture of a medicament, for example, for minimising or treating excitotoxicity.

In the context of this application substantially pure is intended to mean 90% purity or greater such as 95% purity, particularly 98% purity, especially 99% purity, for example as assessed by HPLC analysis.

The invention also extends to employing at least two compounds of Formula (I) in the various aspects of the invention described herein.

Pharmaceutical formulations include those suitable for oral, parenteral (including subcutaneous, intradermal, intramuscular, intravenous and intraarticular), inhalation (including use of metered dose pressurised aerosols, nebulisers or insufflators), intranasal, rectal and topical (including dermal, buccal, sublingual and intraocular) administration. The most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carrier or finely divided solid carriers or both and then, if necessary, shaping the product into the desired formulation.

Formulations of the present invention suitable for oral administration may be presented as discrete units such as capsules such as gelatine or hydroxypropyl methylcellulose (HPMC) capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a paste.

When compounds of Formula (I) are formulated as capsules preferably the compound is formulated with one or more pharmaceutically acceptable carrier such as starch, lactose, microcrystalline cellulose, silicon dioxide and/or a cyclic oligosaccharide such as cyclodextrin.

Additional ingredients may include lubricants such as magnesium stearate and/or calcium stearate.

Suitable cyclodextrins include α-cyclodextrin, β-cyclodextrin, γ-cyclodextrin, dimethyl-β-cyclodextrin, 2-hydroxyethyl-β-cyclodextrin, 2-hyroxypropyl-cyclodextrin, 3-hydroxypropyl-β-cyclodextrin and tri-methyl-β-cyclodextrin. More preferably the cyclodextrin is hydroxypropyl-β-cyclodextrin.

Tablets may be prepared by compression or moulding, optionally with one or more accessory ingredients. Compressed tablets may be prepared by compressing in a suitable machine the active ingredient in a free-flowing form such as a powder or granules, optionally mixed with a binder, lubricant such as magnesium stearate or calcium stearate, inert diluent or a surface active/dispersing agent. Moulded tablets may be made by moulding a mixture of the powdered compound moistened with an inert liquid diluent, in a suitable machine. The tablets may optionally be coated, for example, with an enteric coating and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient and which may include suspending agents and thickening agents. Preferably a parenteral formulation will comprise a cyclic oligosaccharide such as hydroxypropyl-β-cyclodextrin. The formulations may be presented in unit-dose or multi-dose containers, for example sealed ampoules and vials, and may be stored in a freeze-dried (lyophilised) condition requiring only the addition of the sterile liquid carrier, for example saline or water-for-injection (WFI), immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the one or more compounds of the invention and a suitable powder base (carrier substance) such as lactose or starch. Use of lactose is preferred. Each capsule or cartridge may generally contain between 20 μg-10 mg of a compound Formula (I) optionally in combination with another therapeutically active ingredient. Alternatively, the compound or compounds of the invention may be presented without excipients. Packaging of the formulation may be for unit dose or multi-dose delivery.

Spray compositions for topical delivery to the lung by inhalation may, for example be formulated as aqueous solutions or suspensions or as aerosols suspensions or solutions delivered from pressurised packs, such as a metered dose inhaler, with the use of a suitable liquefied propellant. Suitable propellants include a fluorocarbon or a hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. When the excipient is lactose it will typically be present as milled lactose, wherein not more than 85% of lactose particles will have a MMD of 60-90 μm and not less than 15% will have a MMD of less than 15 μm.

Formulations for intranasal administration include mucoadhesive nano-emulsions. Preferably, an intranasal formulation will comprise a mucoadhesive polymer such as Chitosan, and may optionally include additives such as an oil, surfactant, cosurfactant, and combinations thereof. Suitable oils include oleic acid, which enhance transmembrane delivery. Suitable surfactants and cosurfactants include Tween 80, PEG, Labrasol, Carbitol, Tanscutol HP, Cremophore EL, Tween 20, Span 20, ethyl alcohol. Intranasal formulations may be prepared as a sterile powder or suspension of the kind previously described and may contain a preservative.

Medicaments for intranasal administration desirably have a controlled particle size. The optimum particle size for intranasal delivery is usually less than about 1 μm, preferably less than about 500 nm, more preferably less than about 200 nm.

It should be understood that in addition to the ingredients particularly mentioned above, the formulations of this invention may include other agents conventional in the art having regard to the type of formulation in question, for example those suitable for oral administration may include flavouring agents.

The compounds and pharmaceutical formulations according to the invention may be used in combination with or include one or more other therapeutic agents, for example anti-inflammatory agents for minimisation or treatment of neuroinflammation.

Examples may include corticosteroids and NSAIDs. Suitable corticosteroids, which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-17α-[(2-furanylcarbonyl)oxy]-l 1 β-hydroxy-16α-methyl-3-oxo-androsta-1,4-diene-17β-carbothioic acid S-fluoromethyl ester, 6α,9α-difluoro˜l lβ-hydroxy-16α-methyl-3-oxo-l 7α-propionyloxy-androsta-l,4-diene-l 7β-carbothioic acid 5″-(2-OXo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide and butixocort propionate. Preferred corticosteroids include fluticasone propionate, and 6α,9α-difluoro-17α-[(2-furanylcarbonyl)oxy]-11 β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-l lβ-hydroxy-16α-methyl-3-oxo-androsta-l,4-diene-17β-carbothioic acid S-fluoromethyl ester.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors or cytokine synthesis.

The co-administration of active ingredients may be simultaneous or sequential. Simultaneous administration may be affected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

In one embodiment the formulation is an oral formulation, more preferably a capsule formulation.

Preferably the capsule formulation will comprise consist essentially of or consist of a compound of Formula (I) and silicon dioxide.

Preferably the capsule will be a HPMC capsule.

In a preferred embodiment the formulation is a suppository or enema, which can be used to direct the active ingredient more closely to the disease affected area of the body.

Formulations for rectal administration may be presented as a suppository with carriers such as cocoa butter or polyethylene glycol, or as an enema wherein the carrier is an isotonic liquid such as saline. Additional components of the formulation may include a cyclic oligosaccharide, for example, a cyclodextrin, as described above, such as hydroxypropyl-β-cyclodextrin, one or more surfactants, buffer salts or acid or alkali to adjust the pH, isotonicity adjusting agents and/or antioxidants.

In one particularly preferred embodiment, the compound of Formula (I) is provided in the form of a suppository, pessary or intra urethral device in a composition that includes an oleaginous suppository base. In this embodiment, the base is formulated so as to ensure that the bulk of the compound of Formula (I) does not partition from the base.

Generally the base has a solvent power for the compound of Formula (I) enabling at least partial, preferably complete dissolution of the isoflavonoid in the base.

The base may be comprised of, or consist of an oil or fat.

In one embodiment the base includes saturated fatty acids in an amount of 50 to 65% w/w base. Stearic acid may be included in an amount of 25 to 40% w/w base. Palmitic acid in an amount of 25 to 30% w/w base. Longer chain saturated fatty acids such as myristic, arachidic and lauric acid may be included in an amount of <2% w/w base.

In one embodiment, oleaginous bases that include unsaturated fatty acids in an amount of 35 to 50% w/w base are preferred. Monounsaturated fatty acid may be included in an amount of 30 to 45% w/w base. Oleic acid may be included in an amount of 30 to 40% w/w base. Polyunsaturated fatty acids such as linoleic and alpha linolenic acid may be included in an amount of 0 to 5% w/w base.

Theobroma oil (cocoa butter) has been a traditional base in a suppository because of: (a) its non-toxic and non-irritant nature, and (b) its low melting point, meaning that it readily dissolves at body temperature when placed within a bodily cavity, However, it is increasingly being replaced for a number of reasons. One reason is its variability in composition, a consequence of its natural origins; Theobroma oil also is polymorphic, meaning it has the ability to exist in more than one crystal form. Another is that the formulated product needs to be kept refrigerated because of its low melting point, rendering it unsuitable in tropical regions. This has led to a number of substitute products offering a range of advantages over Theobroma oil such as greater consistency, decreased potential for rancidity, and greater ability to tailor phase transitions (melting and solidification) to specific formulation, processing, and storage requirements.

Nevertheless, Theobroma oil or a fatty base with similar composition and physicochemical properties has been found to be a preferred embodiment of the invention.

Typically the oleaginous base comprises a predominance of (>45% w/w base) of saturated fatty acids. Preferably the oleaginous base is Theobroma oil (cocoa butter) or an oil fraction or derivative or synthetic version thereof having a saturated fatty acid profile substantially the same as, or identical to the fatty acid profile of Theobroma oil.

Other examples of oils that may be used to provide or obtain fatty acids useful as bases include those obtainable from natural sources such as canola oil, palm oil, soya bean oil, vegetable oil, and castor oil. Oils derived from these sources may be fractionated to obtain oil fractions containing saturated fatty acids.

The base may be formed or derived from a hard fat, butter or tallow.

A base may comprise esterified or non-esterified fatty acid chains. The fatty acid chains may be in the form of mono, di and triglycerides, preferably of saturated fatty acid chains of C9-20 chain length. The base may comprise additives including PEG, PEG monoesters, PEG monostearate, PEG diesters, PEG distearate, polysorbate esters, and combinations thereof.

A suppository base may be formed from synthetic oils or fats, examples including Fattibase, Wecobee, Witepsol (IOI Oleo GmbH, Germany), Suppocire (Gattefossé, France), Hydrokote, Subanal (Dott. Bonapace, Italy) and Dehydag.

The proportion of the oleaginous suppository base in the final product is a function of the dosage of active pharmaceutical ingredient and the presence of other pharmaceutical or inert ingredient (if any) but may be provided by way of example in an amount of about 1 to 99% w/w formulation.

The compositions for rectal, vaginal or urethral application may be prepared as follows. The compound of Formula (I) is contacted with a suppository base (as described above) in molten form in conditions enabling at least partial, preferably complete or substantially complete dissolution of the compound of Formula (I) in the base. This solution is then poured into a suitable mould, such as a PVC, polyethylene, or aluminium mould. For example, the compound of Formula (I) may be contacted with the base at a temperature of from about 35° C. to about 50° C. and preferably from about 40° C. to about 44° C. The compound of Formula (I) can be milled or sieved prior to contact with the base. In one embodiment, the conditions provided for manufacture, and formulation or device formed from same, enable at least, or provide at least, 50%, preferably 60%, preferably 70%, preferably 80%, preferably 90%, preferably 95% of the isoflavonoid for a given dosage unit to be dissolved in the dosage unit. In these embodiments, no more than 50% of the isoflavonoid for a given dosage unit, preferably no more than 40%, preferably no more than 30%, preferably no more than 20%, preferably no more than 10%, preferably no more than 5% of isoflavonoid for a given dosage unit may be in admixture with, (i.e. undissolved in) the suppository base of the dosage unit.

Optionally the suppositories, pessaries or intra-urethral devices may be coated, prior to packing, for example with cetyl alcohol, macrogol or polyvinyl alcohol and polysorbates to increase disintegration time or lubrication or to reduce adhesion on storage.

One or more sample suppositories, pessaries, or intra-urethral devices from each batch produced are preferably tested by the dissolution method of the present invention for quality control. According to a preferred embodiment, a sample from each batch is tested to determine whether at least about 75 or 80% by weight of the base dissolves within 2 hours.

Typically the suppository, pessary or like device according to the invention is substantially hydrophobic or lipophilic throughout and does not contain a hydrophilic substance such as hydrophilic carrier or pharmaceutical active, or hydrophilic foci or region formed from the ligation or complexing of the isoflavonoid to or with another pharmaceutical compound, carrier or excipient.

The total weight of the suppository preferably ranges from about 1500 mg to about 3000 mg, preferably 1750 mg to about 2500 mg. In another embodiment the total weight of the suppository preferably ranges from about 2250 mg to about 2700 mg, and more preferably from about 2250 to about 2500 mg. According to one embodiment, the suppository has a total weight ranging from about 2300 mg to about 2500 mg.

The suppository or pessary is preferably smooth torpedo-shaped.

The melting point of the suppository or pessary is generally sufficient to melt in the patient's body, and is typically no more than about 37° C.

In one particularly preferred embodiment there is provided:
a kit including:
   a plurality of suppositories sufficient in number to provide an individual with a suppository once daily, or twice daily, for a period of 30 to 90 days, preferably 30 to 60 days, preferably 30 days
   each suppository including:
      400 mg, 800 mg, or 1,200 mg of Compound (1);

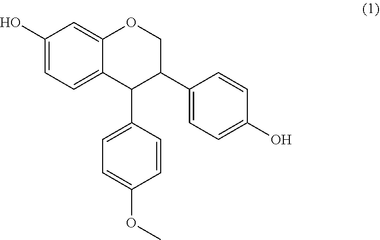 [SMILES: COc1ccc(C2c3ccc(O)cc3OCC2c2ccc(O)cc2)cc1]

a suppository base in the form of cocoa butter;
wherein the suppository base in provided an amount of 1-99% w/w of the suppository,
the kit further including:
   written instructions to provide the suppository once daily, or twice daily for a period of 30 to 90 days, preferably 30 to 60 days, preferably 30 days.

C. Indications and Treatment Methods

The term “excitotoxicity”, unless otherwise indicated herein, refers to a pathological process whereby excitotoxins, such as glutamate, excessively stimulate neuron cells leading to cell damage or death, the latter predominantly by apoptosis.

Excitotoxicity can sometimes be the primary tissue insult leading to injury and formation of a lesion. One example is exposure of brain tissue to exogenous excitotoxins.

More commonly, excitotoxicity is a secondary tissue insult, i.e. an insult arising after an initial tissue insult. One example is the excitotoxicity that follows tissue infarction. Thus excitotoxicity associated with ischemia may lead to the exacerbation of the formation of an infarct, for example in terms of the surface area or volume of the infarct lesion.

There are a number of examples of excitotoxicity that appear to follow an initial insult, and in particular a range of chronic neurodegenerative disorders including Alzheimer's disease, ALS, multiple sclerosis, certain seizure disorders, Parkinson's disease and Huntington's disease. Certain psychiatric, learning or behavioural disorders are also associated with excitotoxicity including autism and Schizophrenia.

The term “condition associated with excitotoxicity” generally refers to conditions in which excitotoxicity tends to follow or accompany an initial insult, examples being acute conditions such as reperfusion/ischemic injury and chronic conditions including the neurodegenerative, psychiatric and learning and behavioural disorders mentioned above. A case history of Transient Ischaemic Attacks (TIAs) is also indicative of progressive excitotoxicity in the brain. In some embodiments, particularly those relating to chronic conditions including neurodegenerative, psychiatric and learning and behavioural disorders, the initial insult, aetiological agent or cause of injury may not be known.

The term “minimising excitotoxicity” generally refers to minimising neuronal damage or death arising from excessive cell stimulation.

The term “preventing” or “inhibiting excitotoxicity” generally refers to providing conditions to a patient so that excessive stimulation of neuronal cells that would otherwise lead to excitotoxic damage is prevented or otherwise minimised. In some embodiments, excitotoxicity is prevented or inhibited by providing a compound according to Formula I prior to induction of excitotoxicity.

The term “treating excitotoxicity” general refers to the minimisation of excitotoxic stimulation of neurons so as to effectively provide a therapeutic benefit to an individual or minimisation of a condition associated with excitotoxicity. The treatment does not necessarily result in the ablation of excitotoxicity.

Excitotoxicity may be observed symptomatically and may also be diagnosed on a cellular or molecular basis. Glutamate levels could be measured in biological fluids, such as plasma and cerebrospinal fluid. Increased levels of the amino acid have been demonstrated in cerebrovascular diseases, amyotrophic lateral sclerosis (ALS) and AIDS dementia complex (ADC). In the case of stroke and ADC, glutamate levels correlated with disease severity. Molecular and biochemical markers of glutamatergic transmission can be assessed in peripheral tissues, such as platelets and fibroblasts, which physiologically express the three major glutamate transporters. Peripheral markers of excitotoxicity in chronic disease including Alzheimer's and Parkinson's disease and ALS are also known [Facheris et al. 2004 J Alzheimers Disease 6:177-184].

In a particularly preferred embodiment, there is provided a method of minimising or treating reperfusion/ischemia related injury such as stroke. The method includes the step of administering a compound according to Formula I, preferably Compound 1, to an individual requiring the treatment. Generally the compound is administered immediately post injury, preferably within 48 hours, preferably within 24 or 12 hours, most preferably within 6 hours or less post injury. The compound may be given intravenously. Alternatively, the compound may be given intra ventricularly. The compound may be given in an amount of about 15 to 30 mg per kg per day or less, for example, 10 to 20 mg per kg per day, such as 1 to 10 mg per kg per day of the body weight of the patient. Typically the compound is given for a period of no longer than about 10 days, preferably 5 to 7 days or less.

In another embodiment there is provided a method of treating a neurodegenerative disease or condition. The disease or condition may be selected from the group consisting of Alzheimer's disease, Parkinson's disease, multiple sclerosis, epilepsy, Huntington's disease and motor neuron disease. Other conditions include those characterized by neuro-fibrillar tangles and/or plaques. In these embodiments, a compound according to Formula I, preferably compound 1 is given one to four times daily, preferably once or twice daily and preferably so as to provide systemic delivery. A unit dose may include from 50 to 500 mg of a compound of Formula I, preferably compound 1, more preferably 200 to 400 mg, for example, about 250 mg. In another embodiment, a unit dose may include 400 mg, 800 mg, or 1,200 mg of a compound of Formula I, preferably compound 1. In a particularly preferred embodiment the compound is given in the form of a suppository in a form described herein. The compound may be given in an amount of about 15 to 30 mg per kg per day or less, for example, 10 to 20 mg per kg per day, such as 1 to 10 mg per kg per day of the body weight of the patient. Typically, the compound is given chronically.

Ultimately, the dose prescribed and intervals at which the prescribed dose is taken will be at the discretion of the physician with responsibility for the patient.

In the embodiments described herein, the individual is typically a human, although other mammals include companion animals such as cats and dogs and other mammalian domestic pets. In an particular embodiment, the companion animal, such as cat or dog requires treatment for seizure or stroke or brain or spinal trauma.

### EXAMPLES

### Example 1 In Vitro Block of Ca²⁺ entry in rHEK293-GCaMPg cells with Nx-1 and Nx-2

Aim

To determine the capacity of Compound 1 to inhibit intracellular Ca²⁺ store release and/or Ca²⁺ entry (Ca²⁺ mobilisation).

Methods

Untransfected human embryonic kidney 293 (HEK293) cells are a model for neuronal cell Ca²⁺ signalling via Gq type G protein-coupled receptors (such as the metabotropic glutamate receptors). These cells exhibit robust G protein (Gq) coupled activation of phospholipase C—inositol trisphosphate (IP₃) production [Jiefei T et al., 1999 Biochemical Journal, 343, 39-44], where inositol trisphosphate (IP₃) activates the IP₃ receptors, which are ion channels that allow Ca²⁺ to exit into the cytoplasm from the endoplasmic reticulum. The current study utilised a custom-developed recombinant human embryonic kidney 293 (rHEK293) cell line stably over-expressing a genetically encoded Ca²⁺ reporter (GCaMP5g—after [Akerboom J et al., 2012 Journal of Neuroscience, 32(40): 13819-13840]). This enabled imaging of the Ca²⁺ store release under conditions where Carbachol (muscarinic receptor agonist)—induced Ca²⁺ store release via the endogenous M3 muscarinic receptor—was delivered in a solution (in mM: NaCl 145; KCl 5.8; MgCl₂ 0.9; HEPES 10, NaH₂PO₄ 0.7, d-glucose 5.6; pH 7.2-7.4, osmolarity 310-330 mOsm/L), lacking Ca²⁺, to the wells containing the adherent rHEK293-GCaMP5g cells, using a FlexStation 3 multi-modal microplate reader (Molecular Devices). This paradigm, with sustained Carbachol presentation, results in extrusion of the cytosolic Ca²⁺ from the cell via plasma membrane Ca²⁺ pumps, alongside Ca²⁺ store depletion. When Ca²⁺ is then added to the media, a Ca²⁺ signal is detected that reflects Ca²⁺ entry into the cells via Ca²⁺ entry pathways [Elliott, 2001 Cell calcium, 30, 73-93].

This study investigated the effect of Nx-1 and Nx-2 on the Ca²⁺ re-entry in the rHEK293-GCaMP5g cells using the FlexStation 3 plate reader in ‘Flex Read’ configuration (485 nm excitation, 510 nm emission, 6 flashes per sample, medium detector sensitivity, 37° C., 780 sample points at 1.54 s intervals). The cells were seeded into the 96 well plates in DMEM media with 10% foetal calf serum supplement. The cells approached confluence at ˜66 hours at which stage the cells were washed with the Ca²⁺-free solution, and then the drug was added in Ca²⁺ free solution (80 μl) and the plate was then placed into the FlexStation plate reader (37° C.) and rested for 30 minutes prior to imaging. The Carbachol (final concentration 100 μM in Ca²⁺ free solution; drug at specified concentration) was added 180 seconds after the start of the Ca²⁺ imaging read. The Ca²⁺ entry was triggered at 480 seconds by adding 10 μl of Carbachol in Ca²⁺ solution with the drug (final concentrations: 100 uM Carbachol, Ca²⁺1.3 mM; drug at specified concentration). The Ca²⁺ imaging proceeded for a total duration of 20 minutes. Controls utilized DMSO carrier at corresponding dilution used with the drug at specified concentrations. Data analysis was based on the areas under the curves (RFU—relative fluorescence Units), with statistical analysis using Sigmaplot (Systat software, version 12.0) and graphics produced using Graphpad Prism (Graphpad Software, version 7.02).

Nx-1 has the following structure:

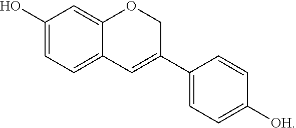 [SMILES: Oc1ccc(C2=Cc3ccc(O)cc3OC2)cc1]

Nx-2 is a 1:1 mixture of the pair of enantiomers of Compound 1(a) and Compound 1(b) as described above.

Results

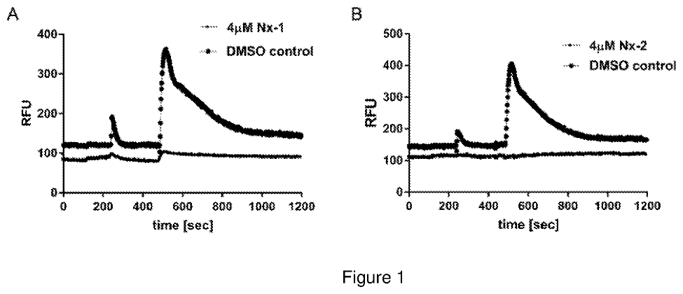

Representative traces of the cytosolic Ca²⁺ levels in HEK293 cells expressing the genetically encoded Ca²⁺ reporter (GCaMP5G) are shown in FIG. 1. Data was obtained from a FlexStation 3 (Molecular Devices) multimodal microplate reader at 485 nm excitation, 510 nm emission.

Nx-1 (4 μM) blocked carbachol (100 μM)—induced Ca²⁺ store release (in Ca²⁺ free solution at 200 s (FIG. 1(A). This is via activation of the endogenous M3 G protein coupled muscarinic receptor activating (via Gq) phospholipase C (PLC), to produce inositol trisphosphate (IP₃). IP₃ activates the IP₃ receptor channels on the endoplasmic reticulum, resulting in release of stored Ca²⁺ into the cytoplasm (increase in fluorescence signal seen in the Dimethysulfoxide (DMSO, 0.04%) carrier control (upper trace). The M3 receptor signalling via PLC also produces diacylglycerol (DAG), which enables endogenous Ca²⁺ entry channel activation (e.g. TRPC channels). This is evident at 500 s (DMSO carrier trace) when 1.3 mM Ca²⁺ is restored to the wells while the Carbachol concentration is maintained.

Nx-1 also blocked Ca²⁺ re-entry. Example of the block of Gq—PLC—IP3 mediated Ca²⁺ store release by Nx-2 (4 μM), and of Ca²⁺ re-entry, is shown in FIG. 1B.

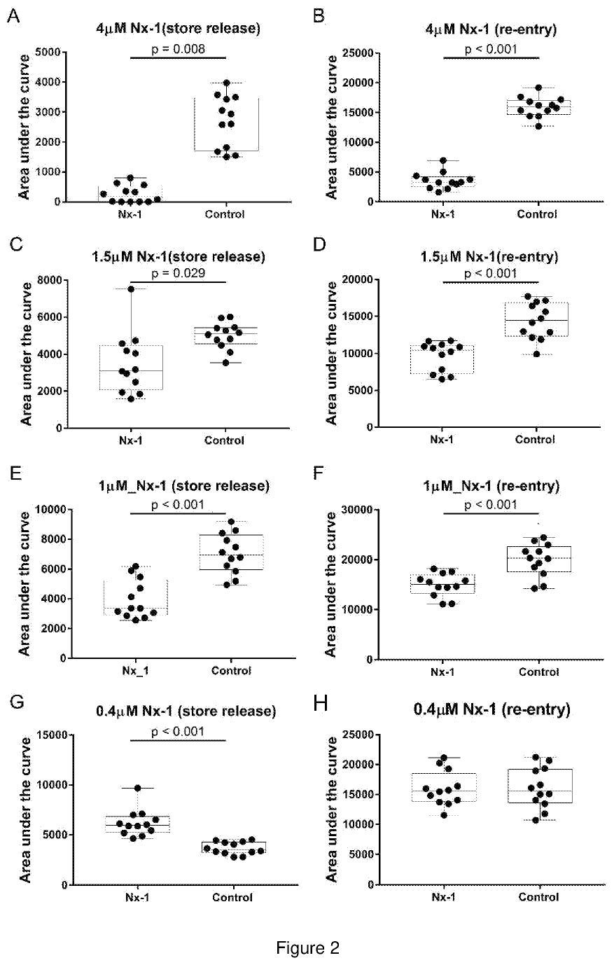

Ca²⁺ store release and Ca²⁺ re-entry by Nx-1 as described in FIG. 1 for HEK293 cells expressing the GCaMP5G Ca²⁺ reporter are described in FIG. 2 in context of areas under the curve measurements for RFU (relative fluorescence units—FlexStation 3; store release response measured between 180 s-350 s; Ca²⁺ re-entry measured from 460 s-700 s).

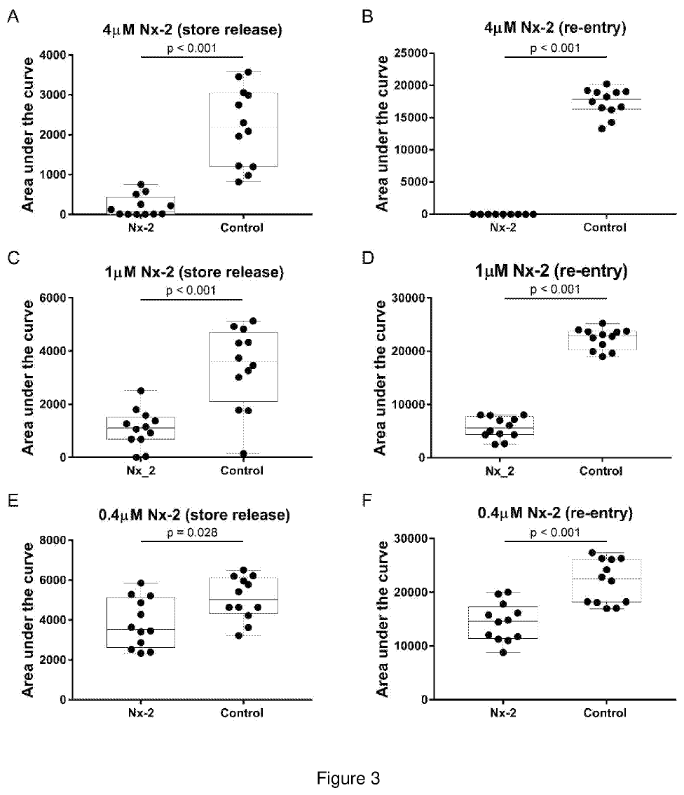

The block of Ca²⁺ store release and Ca²⁺ re-entry by Nx-2 as described in FIG. 1 for HEK293 cells expressing the GCaMP5G Ca²⁺ reporter are described in FIG. 3. Here the responses are measured as areas under the curve for RFU (relative fluorescence units—FlexStation 3; store release response measured between 180 s—350 s; Ca²⁺ re-entry measured from 460 s-700 s).

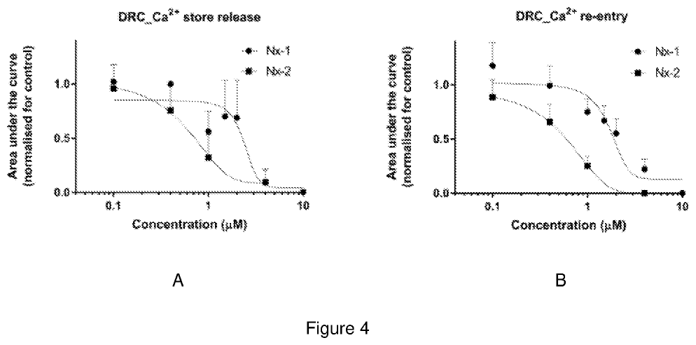

The inhibition of Ca²⁺ store release and Ca²⁺ re-entry by Nx-1 and Nx-2 is described in the context of dose response curves in FIG. 4. For Ca²⁺ store release, data for 0.4 μM of Nx-1 is normalised to 1.0. Ca²⁺ store release is virtually blocked at 4 μM for both Nx-1 and Nx-2, and the data points therefore overlay.

The EC₅₀ is ˜1.5 μM for Nx-1 and ˜600 nM for Nx-2 for both Ca²⁺ store release and re-entry. Data are areas under the curves analysed using relative fluorescent units, normalised against the average of the DMSO control data (n=8-12 per concentration, mean±std.dev.).

In summary, the data shows that Nx-1 and Nx-2 block Ca²⁺ store release and Ca²⁺—re-entry.

### Example 2 In Vivo Model of Block of Ca²⁺ Mobilisation by Compound 1

Aim

To determine the capacity of Compound 1 to mitigate neuronal injury by blocking of Ca²⁺ mobilisation associated with brain ischemia in vivo.

Methods

All in vivo experiments were performed with University of New South Wales (UNSW) Sydney Animal Care and Ethics Committee approval. To undertake this randomized blinded photothrombotic study, male and female mice (C57BI/6J) aged 2-4 months (20-30 g) were anaesthetised with a 4% isoflurane induction (on O₂) and fixed onto a stereotaxic frame. Anaesthesia was maintained at 1.5% isoflurane for the rest of the procedure. The animals were constantly monitored for their heart rate and oxygen saturation with their body temperature maintained at 37±1° C. (Kent Scientific Physiology Suite system). An ophthalmological ointment was applied to the eyes for corneal protection. Analgesia Temgesic (Buprenorphine; 0.15 mg/kg; 0.32 mg/ml) was administered intramuscularly, using a 27G needle. The skull was shaved over the target cerebral cortex region and a local anaesthetic Lignocaine was injected subcutaneously (27 needle; 20 mg/ml). A midline incision was made through the skin to expose the cranium and the periosteum was removed. The surface of the cranium was made lucid by applying ophthalmological gel over the surface. The fibre-optic coupled green LED (532 nm; ˜1 mW; 1 mm diameter light guide, Thorlabs, USA) was placed over the left hemisphere of cerebral cortex (3 mm rostral to lambda; 2 mm from the midline) followed by injection of photosensitising dye Rose Bengal (50 mg/kg) with a 29G needle via the tail vein. The skull was illuminated for 5 minutes to trigger the localized thrombus formation at the target region of cerebral cortex. The skin was then sutured and the isoflurane anaesthesia was discontinued, while providing 100% oxygen until the mice regained consciousness. Mice were injected with saline (intraperitoneal, 250 μl) during the recovery phase for rehydration. The mouse was maintained on the rectal probe feedback controlled heating pad in a recovery cage until ambulatory (˜20 min) and then the animal was placed back in its cage with food and water. The mice were monitored for signs of post-operative stress on a daily basis for the five day treatment period. Mice were administered with either the carrier or Nx-2 drug (100 mg/kg) via a rectal suppository, 45 minutes after the photothrombotic lesion, and then every 24 hours for a further 4 days in the conscious mouse. At 5 days post-infarct, the mice were euthanized via an intraperitoneal injection of Lethabarb (100 mg/kg; 27 G needle), following which the mice were transcardially perfused with 4% paraformaldehyde (PFA) in phosphate buffer (0.1 M, pH 7.4). The brains were dissected out and post-fixed for overnight in 4% PFA at 4° C. The brains were cryoprotected in 10% and 30% sucrose made in 0.01 M PBS solutions overnight respectively. Images of the surface infarct were captured on a dissecting microscope. Following cryoprotection, the brains were sectioned coronally (50 μm thick) and kept in 0.01 M PBS until they were imaged under the darkfield microscope. The Image J (NIH) software was used to measure the surface area of the infarct and darkfield sections enabled measurement of the cross sectional area of the infarct (Image J, NIH). Total infarct volume was quantified by summation of areas of infarct from darkfield sections integrated with thickness of the tissue. Descriptive statistics, ANOVA and t-tests were performed to detect the significance difference among the treatment groups using Sigmaplot (Systat software, version 12.0) after establishing normal distribution of the data (alpha=0.05).

Results

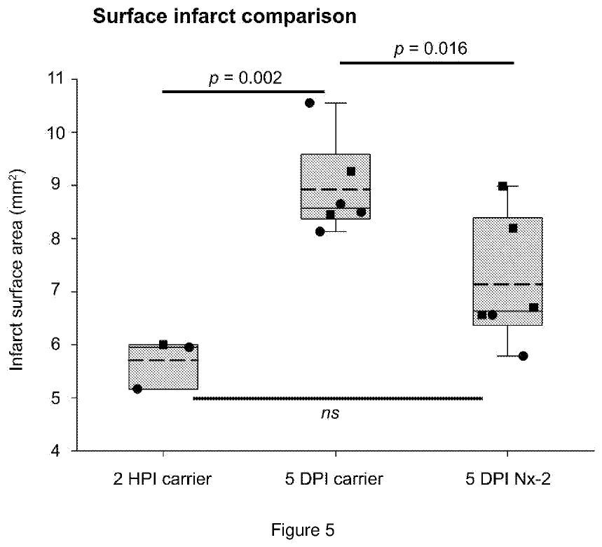

The cerebral cortex surface infarct area of the core reference lesion at 2 hours post injury (2HPI, n=3) compared with the lesion at 5 days post injury (5 DPI) in mice treated with carrier only (n=6) or Nx-2 drug (100 mg/kg; n=6) is shown in FIG. 5. The box plot shows the 25^{th} and 75^{th} percentile boundaries with the error bars indicating the 95% spread. The individual data points of surface infarct area were overlaid on the box plot and the dashed lines represents the mean values. There is a significant difference between the surface infarct area at 2 HPI and 5 DPI carrier treatments (p=0.002) and for 5 DPI carrier versus 5 DPI Nx-2 (p=0.016). The 2 HPI represents the size of the core photothrombotic infarct arising from occlusion of the microvasculature, while the 5 DPI data reflects the expansion of the penumbra. Neuroprotection by Nx-2 is 50% of the penumbra expansion (and is not significantly different from the core infarct surface area (2 HPI control); p=0.064). Statistics represent ANOVA with Holm-Sidak post-hoc comparisons with validation of normal distribution of the data. Circles represent female mice and squares are males.

TABLE 1
Surface infarct area values for carrier only
and Nx-2 at 5 days post infarct

| | | infarct surface |
|---|---|---|
| Mouse ID | Treatment | area (mm²) |
| 63 | Carrier only | 10.56 |
| 61 | Carrier only | 8.13 |
| 58 | Carrier only | 8.45 |
| 55 | Carrier only | 8.65 |
| 52 | Carrier only | 9.27 |
| 47 | Carrier only | 8.50 |
| 67 | Nx-2 (100 mg/kg) | 6.56 |
| 64 | Nx-2 (100 mg/kg) | 6.56 |
| 59 | Nx-2 (100 mg/kg) | 8.99 |
| 57 | Nx-2 (100 mg/kg) | 8.19 |
| 53 | Nx-2 (100 mg/kg) | 5.79 |
| 48 | Nx-2 (100 mg/kg) | 6.70 |

TABLE 2
Core infarct Reference-Infarct Surface Area
measured 2 hours post lesion

| Mouse ID | Treatment | infarct surface area (mm²) |
|---|---|---|
| 51 | Carrier only | 5.16 |
| 50 | Carrier only | 6.00 |
| 49 | Carrier only | 5.95 |

Mean surface area (mm²) for 2 hours post-infarct=5.706±0.271 (n=3, mean±sem).

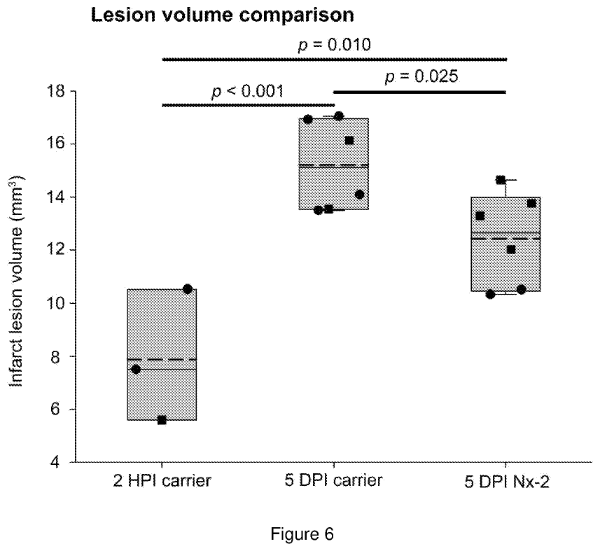

The box plot in FIG. 6 shows the 25^{th} and 75^{th} percentile boundaries with the error bars indicating the 95% percentile. The individual data points of the infarct volumes were overlaid on the box plot and the dashed lines represent the mean values. There is a significant difference between the infarct volume at 2 HPI and infarct volume at 5 DPI carrier treatment (p<0.001); 2 HPI carrier and Nx-2 treatment (p=0.010); 5DPI carrier vs. Nx-2 treatment (p=0.025) (ANOVA, with holm-Sidak post-hoc comparisons). Circles represent female mice and squares are males.

TABLE 3
Infarct volume values for carrier only
and Nx-2 at 5 days post infarct

| Mouse ID | Treatment | infarct volume (mm³) |
|---|---|---|
| 63 | Carrier only | 16.93 |
| 61 | Carrier only | 13.50 |
| 58 | Carrier only | 13.55 |
| 55 | Carrier only | 17.05 |
| 52 | Carrier only | 16.14 |
| 47 | Carrier only | 14.09 |
| 67 | Nx-2 (100 mg/kg) | 13.29 |
| 64 | Nx-2 (100 mg/kg) | 10.34 |
| 59 | Nx-2 (100 mg/kg) | 14.65 |
| 57 | Nx-2 (100 mg/kg) | 12.02 |
| 53 | Nx-2 (100 mg/kg) | 10.51 |
| 48 | Nx-2 (100 mg/kg) | 13.76 |

TABLE 4
Core infarct Reference-Infarct Volume measured 2
hours post lesion

| Mouse ID | Treatment | infarct volume (mm³) |
|---|---|---|
| 51 | Carrier only | 7.50 |
| 50 | Carrier only | 5.59 |
| 49 | Carrier only | 10.53 |

Mean infarct volume for 2 hours post-infarct=7.873±1.439 (n=3, mean±sem).

### Example 3 In Vivo Model of Block of Ca²⁺ Entry by Nx-1

Aim

To determine the capacity of Nx-1 to mitigate neuronal injury by blocking of Ca²⁺ mobilisation associated with brain ischemia in vivo.

Methods

The skull of the mouse (C57BI/6J strain) was exposed to enable fibre optic illumination of a region of the cerebral cortex with green light (532 nm) to trigger localized thrombus formation due to tail vein injection of the photo sensitizing dye Rose Bengal. Macroscopic representation of the photo-thrombotic infarct was assessed 5 days post injury in mice treated with carrier only or with Nx-1 drug. Representative median darkfield images of the coronal brain slice (50 μm thick) from mice treated with carrier only or Nx-1 drug where taken and damaged tissue was assessed.

Results

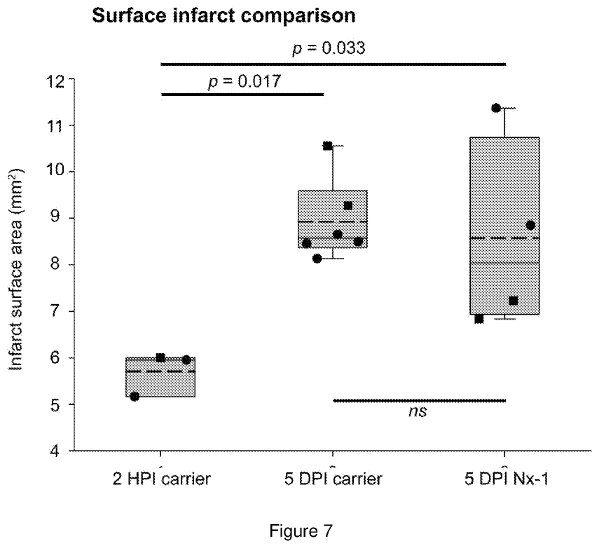

The box plot shows the 25^{th} and 75^{th} percentile boundaries with the error bars indicating the 95% percentile is shown in FIG. 7. The individual data points of the infarct volumes were overlaid on the box plot and the dashed line represents the mean value. There was a significant difference between the infarct volume at 2 DPI and infarct volume at 5 DPI carrier treatment (p=0.004); 2 HPI carrier and Nx-1 treatment (p=0.004); (ANOVA, with holm-Sidak post-hoc comparisons). Infarct volume between 5 DPI carrier treatment and Nx-1 drug is not significantly different (p=0.886). Circles represent females and squares are males.

TABLE 5
Infarct volume values for carrier only and Nx-1 at
5 days post infarct

| Mouse ID | Treatment | infarct volume (mm³) |
|---|---|---|
| 63 | Carrier only | 16.93 |
| 61 | Carrier only | 13.50 |
| 58 | Carrier only | 13.55 |
| 55 | Carrier only | 17.05 |
| 52 | Carrier only | 16.14 |
| 47 | Carrier only | 14.09 |
| 68 | Nx-1 (100 mg/kg) | 11.80 |
| 62 | Nx-1 (100 mg/kg) | 18.94 |
| 60 | Nx-1 (100 mg/kg) | 14.16 |
| 54 | Nx-1 (100 mg/kg) | 16.84 |

TABLE 6
Core infarct Reference-Infarct Volume
measured 2 hours post lesion

| Mouse ID | Treatment | infarct volume (mm³) |
|---|---|---|
| 51 | Carrier only | 7.50 |
| 50 | Carrier only | 5.59 |
| 49 | Carrier only | 10.53 |

Mean infarct volume for 2 hours post-infarct=7.873±1.439 (n=3, mean±sem).

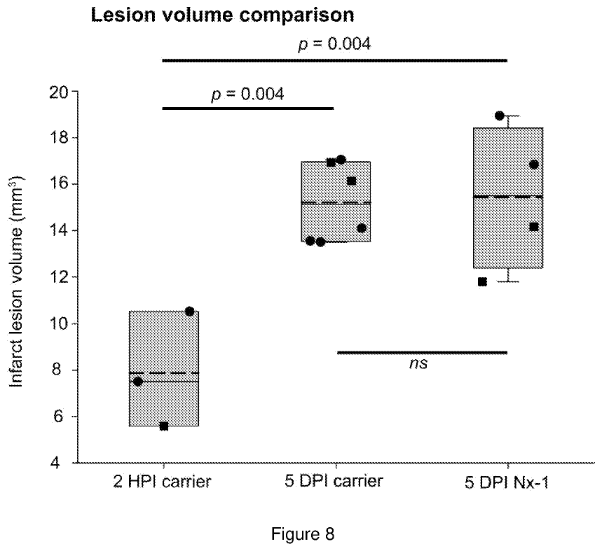

The box plot shows the 25^{th} and 75^{th} percentile boundaries with the error bars indicating the 95% percentile is shown in FIG. 8. The individual data points of the infarct volumes were overlaid on the box plot and the dashed line represents the mean value. There was a significant difference between the infarct volume at 2 HPI and infarct volume at 5 DPI carrier treatment (p=0.004); 2 HPI carrier and Nx-1 treatment (p=0.004); (ANOVA, with holm-Sidak post-hoc comparisons). Infarct volume between 5 DPI carrier treatment and Nx-1 drug is not significantly different (p=0.886). Circles represent females and squares are males.

TABLE 7
Infarct volume values for carrier only
and Nx-1 at 5 days post infarct

| Mouse ID | Treatment | infarct volume (mm³) |
|---|---|---|
| 63 | Carrier only | 16.93 |
| 61 | Carrier only | 13.50 |
| 58 | Carrier only | 13.55 |
| 55 | Carrier only | 17.05 |
| 52 | Carrier only | 16.14 |
| 47 | Carrier only | 14.09 |
| 68 | Nx-1 (100 mg/kg) | 11.80 |
| 62 | Nx-1 (100 mg/kg) | 18.94 |
| 60 | Nx-1 (100 mg/kg) | 14.16 |
| 54 | Nx-1 (100 mg/kg) | 16.84 |

TABLE 8
Core infarct Reference-Infarct Volume
measured 2 hours post lesion

| Mouse ID | Treatment | infarct volume (mm³) |
|---|---|---|
| 51 | Carrier only | 7.50 |
| 50 | Carrier only | 5.59 |
| 49 | Carrier only | 10.53 |

Mean infarct volume for 2 hours post-infarct=7.873±1.439 (n=3, mean±sem).

1. A method for inhibiting Ca²⁺ release and/or Ca²⁺ uptake from a cell in an individual, including the step of providing a compound of general formula (I) to the cell 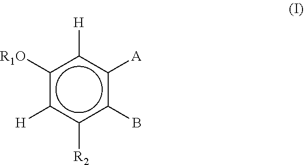
wherein
R₁ is H, or R_{A}CO where R_{A} is C₁₋₂₀ alkyl or an amino acid;
R₂ is H, OH, or R_{B} where R_{B} is an amino acid or OCOR_{A} where R_{A} is C₁₋₂₀ alkyl or an amino acid;
A and B together with the atoms between them form the group: 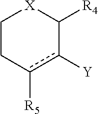
wherein
R₄ is H, COR_{D} where R_{D} is H, OH, C₁₋₂₀ alkyl or an amino acid, CO₂R_{C} where R_{C} is C₁₋₂₀ alkyl, COOH, or CONHR_{E} where R_{E} is H, C₁₋₂₀ alkyl or an amino acid;
R₅ is substituted or unsubstituted aryl or substituted or unsubstituted heteroaryl;
X is O, N or S;
Y is 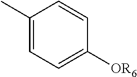
where R₆ is H, or R_{A}CO where R_{A} is C₁₋₂₀ alkyl or an amino acid; and
“ [symbol] ” represents either a single bond or a double bond,
thereby inhibiting Ca²⁺ release and/or Ca²⁺ uptake by the cell in the individual.

2. A method according to claim 1, wherein X is O.

3. A method according to claim 1 or claim 2, wherein R₁ is H.

4. A method according to any one of claims 1 to 3, wherein R₂ is H.

5. A method according to any one of claims 1 to 4, wherein R₄ is H.

6. A method according to any one of claims 1 to 5, wherein R₅ is substituted aryl.

7. A method according to claim 6, wherein R₅ is hydroxy- or alkoxy-substituted aryl.

8. A method according to claim 7 wherein the alkoxy group is methoxy.

9. A method according to claim 8 wherein R₅ is methoxyphenyl.

10. A method according to any one of claims 6 to 9, wherein the substituted aryl has the substituent in the 4-position.

11. A method according to any one of claims 1 to 10, wherein R₆ is H.

12. A method according to claim 1, wherein the compound of formula (I) is

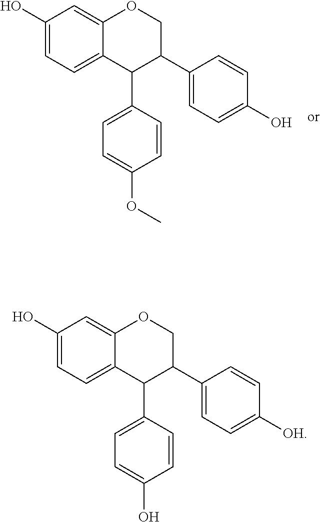 [SMILES: COc1ccc(C2c3ccc(O)cc3OCC2c2ccc(O)cc2)cc1 ; Oc1ccc(C2COc3cc(O)ccc3C2c2ccc(O)cc2)cc1]

13. A method according to claim 1, wherein the compound of formula (I) is

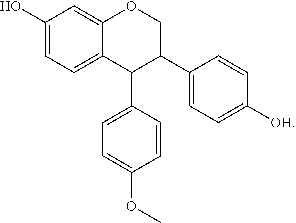 [SMILES: COc1ccc(C2c3ccc(O)cc3OCC2c2ccc(O)cc2)cc1]

14. A method according to claim 13, wherein the compound is compound 1(a).

15. The method according to any one of claims 1 to 14, wherein the compound is formulated as a suppository, pessary or like.

16. The method according to claim 15, wherein the suppository, pessary or like is dissolved in an oleaginous base.

17. The method according to claim 15 or 16, wherein the suppository, pessary or like includes the compound 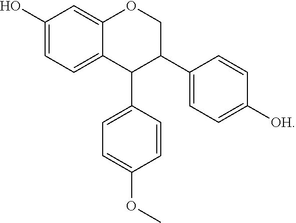 [SMILES: COc1ccc(C2c3ccc(O)cc3OCC2c2ccc(O)cc2)cc1]
in an amount of about 400-1,200 mg.

18. Use of a compound according to formula (I) 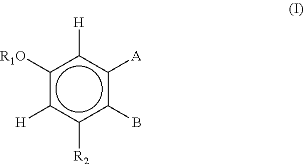
wherein
R₁ is H, or R_{A}CO where R_{A} is C₁₋₂₀ alkyl or an amino acid;
R₂ is H, OH, or R_{B} where R_{B} is an amino acid or OCOR_{A} where R_{A} is C₁₋₂₀ alkyl or an amino acid;
A and B together with the atoms between them form the group: 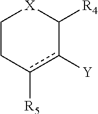
wherein
R₄ is H, COR_{D} where R_{D} is H, OH, C₁₋₂₀ alkyl or an amino acid, CO₂R_{C} where R_{C} is C₁₋₂₀ alkyl, COOH, or CONHR_{E} where R_{E} is H, C₁₋₂₀ alkyl or an amino acid;
R₅ is substituted or unsubstituted aryl or substituted or unsubstituted heteroaryl;
X is O, N or S;
Y is 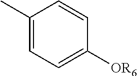
where R₆ is H, or R_{A}CO where R_{A} is C₁₋₂₀ alkyl or an amino acid; and
“ [symbol] ” represents either a single bond or a double bond,
in the preparation of a medicament for inhibiting Ca²⁺ release and/or Ca²⁺ uptake from a cell in an individual.

19. A compound according to formula (I) 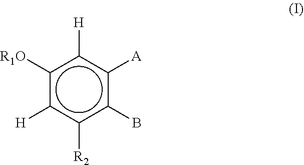
wherein
R₁ is H, or R_{A}CO where R_{A} is C₁₋₂₀ alkyl or an amino acid;
R₂ is H, OH, or R_{B} where R_{B} is an amino acid or OCOR_{A} where R_{A} is C₁₋₂₀ alkyl or an amino acid;
A and B together with the atoms between them form the group: 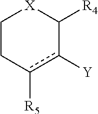
wherein
R₄ is H, COR_{D} where R_{D} is H, OH, C₁₋₂₀ alkyl or an amino acid, CO₂R_{C} where R_{D} is C₁₋₂₀ alkyl, COOH, or CONHR_{E} where R_{E} is H, C₁₋₂₀ alkyl or an amino acid;
R₅ is substituted or unsubstituted aryl or substituted or unsubstituted heteroaryl;
X is O, N or S;
Y is 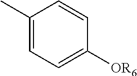
where R₆ is H, or R_{A}CO where R_{A} is C₁₋₂₀ alkyl or an amino acid; and
“ [symbol] ” represents either a single bond or a double bond,
for use in inhibiting Ca²⁺ release and/or Ca²⁺ uptake from a cell in an individual.

20. A method or use according to any one of the preceding claims wherein the cell is a neuron.

21. A method or use according to any one of the preceding claims wherein the Ca²⁺ release and/or Ca²⁺ uptake in the cell is associated with excitotoxicity.

22. The method or use according to claim 21 wherein the excitotoxicity is associated with stroke.

23. The method of claim 22 wherein the compound of Formula 1 is compound 1(a).
